(19) United States
(12) Patent Application Publication
(10) Pub. No.: US 2024/0359996 A1
(43) Pub. Date: Oct. 31, 2024

(54) Low Carbon Emission Optimization for a Combined Ammonia, UAN, and Urea Production Process

(71) Applicant: John Walton Douglas, League City, TX (US)

(72) Inventor: John Walton Douglas, League City, TX (US)

(21) Appl. No.: 18/646,075

(22) Filed: Apr. 25, 2024

Related U.S. Application Data
(60) Provisional application No. 63/498,519, filed on Apr. 26, 2023.

(51) Int. Cl. C01C 1/04; B01F 35/22; B01F 35/221; B01F 35/83; C01B 3/36; C05C 1/00; C05C 9/00; C05G 5/23; C07C 273/10

(52) U.S. Cl. CPC C01C 1/0488; B01F 35/2206; B01F 35/2217; B01F 35/833; C01B 3/36; C01C 1/0417; C05C 1/00; C05C 9/005; C05G 5/23; C07C 273/10; C01B 2203/025; C01B 2203/068; C01B 2203/1235; C01B 2203/82

(57) ABSTRACT

Methods and systems for producing ammonia, urea, and UAN are described herein. The methods can include providing a hydrocarbon feed stock and an oxygen-containing stream and reacting the hydrocarbon feed stock with the oxygen-containing stream to provide a syngas containing carbon dioxide and blue hydrogen. The methods can also include electrolyzing water to provide green hydrogen and blending the blue hydrogen with the green hydrogen to provide a blended hydrogen. The blended hydrogen can be introduced to an ammonia synthesis system to provide an ammonia product, which can be further processed to provide urea and/or UAN. The ratio of green to blue hydrogen in the blended hydrogen can be based on available green credits.

### PRIORITY CLAIM

This U.S. non-provisional patent application claims priority to and the benefit of, under 35 U.S.C. § 119 (c), U.S. Provisional Application No. 63/498,519, filed Apr. 26, 2023, the disclosure of which is incorporated herein by reference in its entirety.

### FIELD

The embodiments described herein relate to the production of ammonia and nitrogen-based fertilizers.

### BACKGROUND

Nitrogen-based fertilizers, such as urea and urea ammonium nitrate (“UAN”), are commonly available macronutrients that are oftentimes necessary to ensure efficient agricultural production. Urea is formed by combining ammonia with carbon dioxide. As such, the production process for urea typically involves the production of carbon dioxide and ammonia. The carbon dioxide component of the urea production process is oftentimes produced by reacting natural gas (methane) with steam in the presence of a catalyst in a process known as steam reforming to provide synthesis gas (“syngas”), which contains carbon dioxide, carbon monoxide, and hydrogen.

The ammonia component of the urea production process is oftentimes produced by the reaction of nitrogen and hydrogen gases under high pressure and temperature in the presence of a catalyst. The Haber-Bosch is a common ammonia production process that uses natural gas as a source of hydrogen gas and air as a source of nitrogen gas.

During the urea production process, ammonia and carbon dioxide are reacted together under high pressure and temperature in the presence of a catalyst to provide ammonium carbamate, which ultimately decomposes into urea and water.

UAN is a liquid fertilizer that contains a combination of nitrogen sources, including urea and ammonium nitrate. UAN is oftentimes produced by combining urea and ammonium nitrate in an aqueous solution.

Currently, production of Urea and UAN relies on the conversion of fossil fuels, including hydrocarbons, resulting in significant emissions of carbon dioxide. What is needed is a method to produce ammonia, urea and UAN in a manner that reduces or eliminates carbon dioxide emissions while ensuring economic feasibility of the production process.

### BRIEF DESCRIPTION OF THE DRAWINGS [if any]

The present invention may best be understood by referring to the following description and accompanying drawings that are used to illustrate embodiments of the invention. In the drawings:

FIG. 1 depicts an illustrative ammonia system, according to one or more embodiments described herein.

FIG. 2 depicts an illustrative integrated ammonia-urea-UAN system, according to one or more embodiments described herein.

FIG. 3 depicts an illustrative flow chart of at least one embodiment of a method for blending grey, blue, and/or green hydrogen, which may be performed by one or more components of the systems of FIGS. 1 and 2, as described herein.

### DETAILED DESCRIPTION

The present disclosure is directed to low carbon emission optimization for a combined ammonia, UAN, and urea production process. In particular, the present disclosure includes systems and methods for producing green hydrogen, blue hydrogen, green ammonia, and/or blue ammonia and methods and systems for optimizing and configuring the production of each to provide reduced emission ammonia, UAN, and urea products, in a manner that reduces the overall cost of such products.

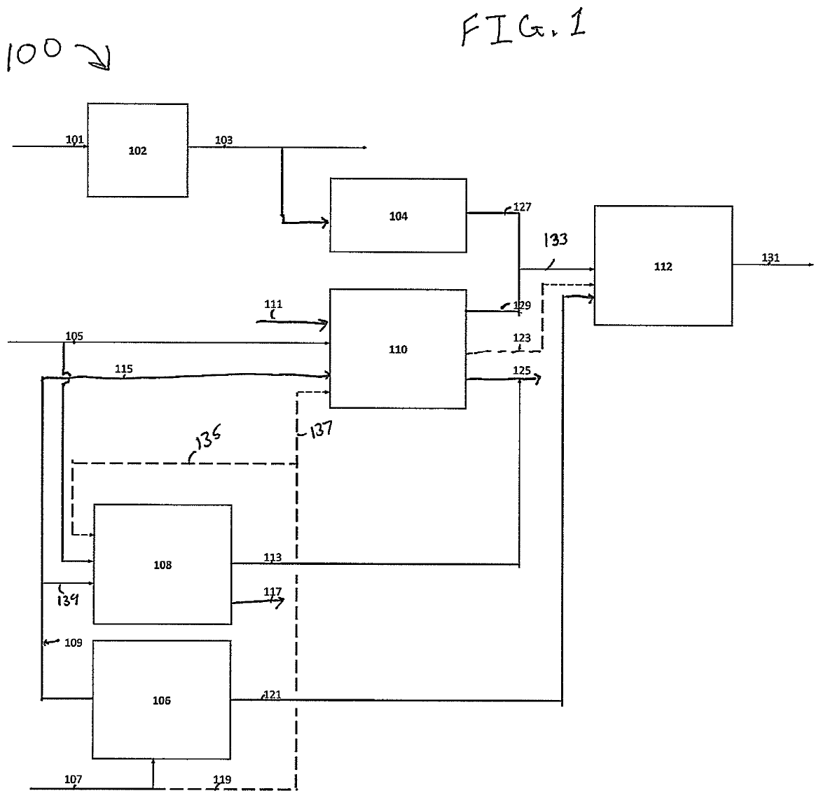

FIG. 1 depicts an illustrative ammonia system 100, according to one or more embodiments. The ammonia system 100 can include an electrolyzer system 104, a synthesis gas (or syngas) generation system 110, and an ammonia synthesis system 112. The ammonia synthesis system 112 can be in fluid communication with the electrolyzer system 104 and the syngas generation system 110. The ammonia system 100 can further include a water treatment system 102 in fluid communication with the electrolyzer system 104. In one or more embodiments, an air separation unit 106 or a power plant 108 or both can be in fluid communication with the syngas generation system 110 and optionally the ammonia synthesis system 112.

In one or more embodiments, the syngas generation system 110 can be or include any suitable system for converting a hydrocarbon material to one or more of hydrogen, carbon dioxide, and carbon monoxide. In one or more embodiments, the syngas generation system 110 can be or include one or more syngas reactors. The one or more syngas reactors can be or include one or more autothermal reformers (“ATR”), one or more steam methane reformers (“SMR”), one or more gasifiers, one or more reforming exchangers, one or more catalytic partial oxidation (“CPOX”) reactors, or one or more partially oxidation (“POX”) reactors, or any combination thereof. In one or more embodiments, the syngas generation system 110 can include one or more primary reformers and one or more secondary reformers. For example, the syngas generation system 110 can include one or more reformers to convert at least a portion of a hydrocarbon in the presence of one or more catalysts, oxidant, heat, flame, steam, or a combination thereof to provide the syngas. In one or more embodiments, the one or more reformers can be arranged in any serial, parallel, or serial/parallel combination. In one or more embodiments, the one or more reforming exchangers can include a KBR Reforming Exchanger System (“KRES”). Additional KRES process conditions, catalysts, and other details can be found in U.S. Pat. Nos. 5,011,625, 5,122,299, 5,362,454, 6,855,272, 7,138,001, and 7,220,505, each of which are fully incorporated by reference herein. Additional reforming exchanger types, catalyst types, process conditions, and other details can be found in U.S. Pat. Nos. 7,074,347 and 6,224,789, which are fully incorporated by reference herein.

In one or more embodiments, the syngas generation system 110 can further include one or more shift reactors, such as a water-gas shift reactor, to increase the hydrogen yield. For example, the resulting product streams, containing steam and carbon monoxide, from the one or more primary reformers and/or one or more secondary reformers can be introduced to one or more shift reactors to convert a gaseous mixture comprising steam and carbon monoxide to hydrogen and carbon dioxide. Suitable water-gas shift reactors are described in U.S. Pat. Nos. 9,340,494 and 9,321,655, which are fully incorporated by reference herein.

In one or more embodiments, the syngas generation system 110 can provide hydrogen to the ammonia synthesis system 112. The hydrogen provided by the syngas generation system 110 can be or include grey hydrogen and/or blue hydrogen. As used herein, the term “grey hydrogen” refers to hydrogen produced from one or more fossil fuels and the term “blue hydrogen” refers to hydrogen produced from one or more fossil fuels where all of the carbon dioxide produced by the conversion of the fossil fuel(s) to hydrogen is captured to be stored or used in other products or processes.

The electrolyzer system 104 can be or include any electrolyzer unit suitable for converting water into oxygen and hydrogen via hydrolysis. Suitable electrolyzer units can include alkaline electrolyzers, proton exchange membrane electrolyzers, or solid oxide electrolyzers, or any combination thereof. The hydrogen provided by the electrolyzer unit can be or include green hydrogen. As used herein, the term “green hydrogen” refers to hydrogen produced by electrolysis of water at least partially powered by one or more of solar energy, wind energy, geothermal energy, hydroelectric energy, wave energy, tidal energy, nuclear energy, biofuels, and biomass gasification.

The ammonia synthesis system 112 can be or include any suitable system for reacting hydrogen and nitrogen to provide ammonia. In one or more embodiments, the ammonia synthesis system 112 can be or include one or more syngas compressors, one or more ammonia converters, and one or more ammonia condensers. In one or more embodiments, the ammonia synthesis system 112 can convert grey hydrogen, blue hydrogen and/or green hydrogen to provide grey ammonia, blue ammonia and/or green ammonia, respectively. As used herein, the term “grey ammonia refers to ammonia produced from nitrogen and grey hydrogen, the term “blue ammonia” refers to ammonia produced from nitrogen and blue hydrogen, the term “green ammonia” refers to ammonia produced from nitrogen and green hydrogen.

In one or more embodiments, the blue hydrogen provided by the syngas generation system 110 can be mixed or blended with the green hydrogen provided by the electrolyzer system 104 prior to and/or during introduction to the ammonia synthesis system 112 to provide a blue-green ammonia. In some embodiments, grey hydrogen provided by the syngas generation system 110 can be mixed or blended with the green hydrogen provided by the electrolyzer system 104 prior to and/or during introduction to the ammonia synthesis system 112 to provide a grey-green ammonia. In one or more embodiments, the blue-green ammonia can contain at least 10 vol % green ammonia, at least 25 vol % green ammonia, or at least 50 vol % green ammonia. In one or more embodiments, the grey-green ammonia can contain at least 10 vol % green ammonia, at least 25 vol % green ammonia, or at least 50 vol % green ammonia.

In some embodiments, the green hydrogen and the blue hydrogen can be separately introduced (e.g., separately introduced as batches) to the ammonia synthesis system 112 to provide green ammonia and blue ammonia. In other embodiments, the green hydrogen, the grey hydrogen, and/or the blue hydrogen can be separately introduced (e.g., separately introduced as batches) to the ammonia synthesis system 112 to provide green ammonia, grey ammonia, and/or blue ammonia.

In one or more embodiments the ammonia system 100 can also include an air separation unit 106. The air separation unit 106 can be any suitable system for separating air to provide highly pure oxygen and nitrogen streams. The air separation unit 106 can be or include cryogenic separation, adsorption air separation, vacuum swing adsorption, or membrane air separation, or combinations thereof. In one or more embodiments, the air separation unit 106 can separate incoming air into a nitrogen stream consisting essentially of nitrogen and an oxygen stream consisting essentially of oxygen.

In one or more embodiments, the ammonia system 100 can also include a power plant 108. The power plant 108 can include a prime mover, such as a gas turbine or a steam turbine or a combination thereof, configured to drive an electric generator to convert mechanical energy from the prime mover to electrical energy.

In operation of the ammonia system 100, a hydrocarbon feed stock via line 105 can be introduced to the to the syngas generation system 110. The hydrocarbon feed stock in line 105 can be or include natural gas (e.g., methane), LPG, refinery gas, naphtha, and the like. The operation of the ammonia system 100 can also include introducing air via line 107 to the air separation unit 106 to provide a nitrogen containing stream via line 121 and an oxygen containing stream via line 109. At least a portion of the oxygen containing stream in line 109 can be introduced to the syngas generation system 110 via line 115 for reaction with the hydrocarbon feed stock in the one or more reactors and/or reformers of the syngas generation system 110 as described herein. Another portion of the oxygen stream in line 109 can be introduced to the power plant 108 via line 139 for use in the gas turbine engine, for example. A portion of the hydrocarbon feed stock can also be introduced to the gas turbine engine as fuel, for example.

In one or more embodiments a portion of the air in line 107 can be introduced to the syngas generation system 110 via line 119. In some embodiments, a portion of the air in line 119 can be directed to the power plant 108 via line 135 for use in the gas turbine, for example. Another portion of the air in line 119 can be introduced to the syngas generation system 110 via line 137 for reaction with the hydrocarbon feed stock in the one or more reactors and/or reformers of the syngas generation system 110 as described herein. The hydrocarbon feedstock and air via line 135 or oxygen via line 139, or a combination thereof, may be subject to a combustion reaction in the gas turbine engine of the power plant 108 to provide a power plant carbon dioxide containing stream via line 113 and a water containing stream via line 117.

In operation of the syngas generation system 110, the hydrocarbon feed stock in line 105 and the oxygen-containing stream in line 115, and optionally, steam in line 111 and/or air in line 137 can be introduced to the one or more syngas reactors as described herein, including but not limited to an ATR reactor having a combustion zone and a reforming zone, a SMR, and/or a CPOX reactor to provide a syngas product.

The hydrocarbon feed stock in line 105 can be or include methane. In one or more embodiments, the hydrocarbon feed stock in line 105 can be at least 75 wt % methane, at least 90 wt % methane, at least 95 wt % methane, or at least 99 wt % methane. The oxygen-containing stream in line 115 can be or include oxygen. In one or more embodiments, the oxygen-containing stream in line 115 can be at least 40 wt % oxygen, at least 50 wt % oxygen, at least 65 wt % oxygen, at least 85 wt % oxygen, at least 90 wt % oxygen, at least 95 wt % oxygen, at least 97 wt % oxygen, or at least 99 wt % oxygen.

In one or more embodiments, the oxygen-containing stream can be mixed with the steam and optionally the air to provide a steam-oxygen mixture. The steam-oxygen mixture can be heated, for example, to a temperature of about 100° C., about 200°° C., or about 300° C. to about 400° C., about 500° C., or about 700° C. or greater to provide a heated steam-oxygen mixture. The heated steam-oxygen mixture can be introduced to the syngas reactor along with the hydrocarbon feed stock. If more than one syngas reactor is utilized, the hydrocarbon feed stock and the heated steam-oxygen mixture can be introduced to such multiple reactors in parallel in any suitable manner. For example, the hydrocarbon feed stock and the heated steam-oxygen mixture can be introduced to two or more of the ATR, the CPOX, and the SMR reactors in parallel to provide the syngas product.

The syngas product can include hydrogen, carbon monoxide, and carbon dioxide in any suitable amounts. To the extent air, for example, via line 137, or nitrogen is otherwise introduced to the syngas reactor(s), the syngas product can also include nitrogen in any suitable amounts. In one or more embodiments the syngas product can be subjected to one or more WGS reactors to provide a treated syngas product containing hydrogen, carbon dioxide, and up to trade amounts of carbon monoxide, for example, less than 1 wt %, less than 0.5 wt %, or less than 0.1 wt % carbon monoxide, and optionally nitrogen.

The syngas product obtained from the syngas reactor(s) can be subjected to a separation step to provide a hydrogen-containing stream via line 129, a carbon dioxide-containing stream via line 125, and an optional nitrogen-containing stream via line 123. The separation step can include subjecting the syngas product to one or more separation steps, such as pressure swing absorption (“PSA”) and/or other types of separation steps, to provide a hydrogen-containing stream via line 129, a carbon dioxide-containing stream via line 125, and an optional nitrogen-containing stream via line 123.

In one or more embodiments, the hydrogen-containing stream via line 129 can be at least 80 wt % hydrogen, at least 90 wt % hydrogen, at least 95 wt % hydrogen, at least 97 wt % hydrogen, or at least 99 wt % hydrogen or more. In one or more embodiments, the hydrogen-containing stream via line 129 can be pure hydrogen. In one or more embodiments, the hydrogen-containing stream via line 129 can be blue hydrogen.

In operation of the electrolyzer system 104, water via line 101 can be introduced to the water treatment system 102 to provide a purified water stream via line 103. At least a portion of the purified water stream via line 103 can be introduced to the electrolyzer system 104. The electrolyzer system 104 can include an electrolyzer configured to use electricity to split water into hydrogen and oxygen gas via electrolysis. The electrolyzer system 104 can include a water storage tank or suitable storage means, an electrolysis cell, and an electrical power source. The water storage tank can be configure to hold a volume of water to be used in the electrolysis process. The electrolysis cell can include two electrodes, an anode and a cathode, separated by a membrane or electrolyte. When an electric current is applied to the electrodes can cause the water molecules in the volume of water to split into hydrogen and oxygen, resulting in the production of hydrogen and oxygen gas.

The electrical power source can be any source of direct current, such as a battery or a rectifier. The voltage and current applied to the electrolysis cell can be controlled to optimize the production of hydrogen gas. The electrical power source can be generated by any suitable source of electricity including the power plant 108. In one or more embodiments, the electricity can be generated by one or more of solar energy, wind energy, geothermal energy, hydroelectric energy, wave energy, tidal energy, nuclear energy, biofuels, and biomass gasification.

The hydrogen gas produced by the electrolyzer system 104 can be collected and stored for use in various applications, such as fuel cells, combustion engines, or chemical synthesis. The oxygen gas can also be collected and used for industrial processes or released into the atmosphere. In one or more embodiments, the hydrogen gas produced by the electrolyzer system 104 can be withdrawn from the electrolyzer system 104 via line 127. In one or more embodiments, the hydrogen gas in line 127 is green hydrogen.

In one or more embodiments, the hydrogen in line 127 can be blended or otherwise mixed with the hydrogen in line 129 to provide a hydrogen blend in line 133. For example, blue hydrogen in line 129 can be blended with the green hydrogen in line 127 to provide the hydrogen blend in line 133.

The blended hydrogen in line 133 can then be introduced to the ammonia unit 112 to provide an ammonia product via line 131. In one or more embodiments, the ammonia unit 112 can include two or more ammonia units (not shown). In one or more embodiments, the green hydrogen in line 127 can be introduced to a first ammonia unit to provide a green ammonia product and the blue hydrogen in line 129 can be introduced to a second, separate ammonia unit to provide a blue ammonia product. In one or more embodiments, the green ammonia product and the blue ammonia product can be blended or otherwise mixed to form a blended ammonia product.

In one or more embodiments, the one or more ammonia units 112 can be single or multi-pass converters using one or more noble metal catalysts, or one or more catalysts based upon ruthenium, such as the ruthenium-based KAAP catalyst available from Kellogg, Brown and Root. In one or more embodiments, the one or more ammonia units 112 can include any reactor intended to operate at elevated pressures and/or temperatures to convert at least a portion of the blended hydrogen in line 133 and the nitrogen in one or more of lines 123 and 121 to ammonia. In one or more embodiments, the one or more ammonia units 112 can include one or more “Split-Flow Ammonia Converters” as described in U.S. Pat. No. 7,081,230, which is fully incorporated by reference herein. In one or more embodiments, the one or more ammonia units 112 can include one or more “Isothermal Ammonia Converters” as described in U.S. Pat. No. 6, 171,570, which is fully incorporated by reference herein. In one or more embodiments, the one or more ammonia units 112 can include one or more “Horizontal Ammonia Converter Adapted for High Activity Catalyst” as described in U.S. Pat. No. 6,132,687, which is fully incorporated by reference herein. In one or more embodiments, the one or more ammonia units 112 can include one or more ammonia converters as described in U.S. patent application Ser. No. 12/107,506, which is fully incorporated by reference herein.

In one or more embodiments, the ammonia concentration of the ammonia product in line 131 can be about 85% wt, about 90% wt, about 95% wt, or about 99.9% wt. In one or more embodiments, the ammonia product in line 131 contain a maximum of about 15% wt, about 10% wt, about 5% wt, or about 0.1% wt of combined hydrogen and nitrogen.

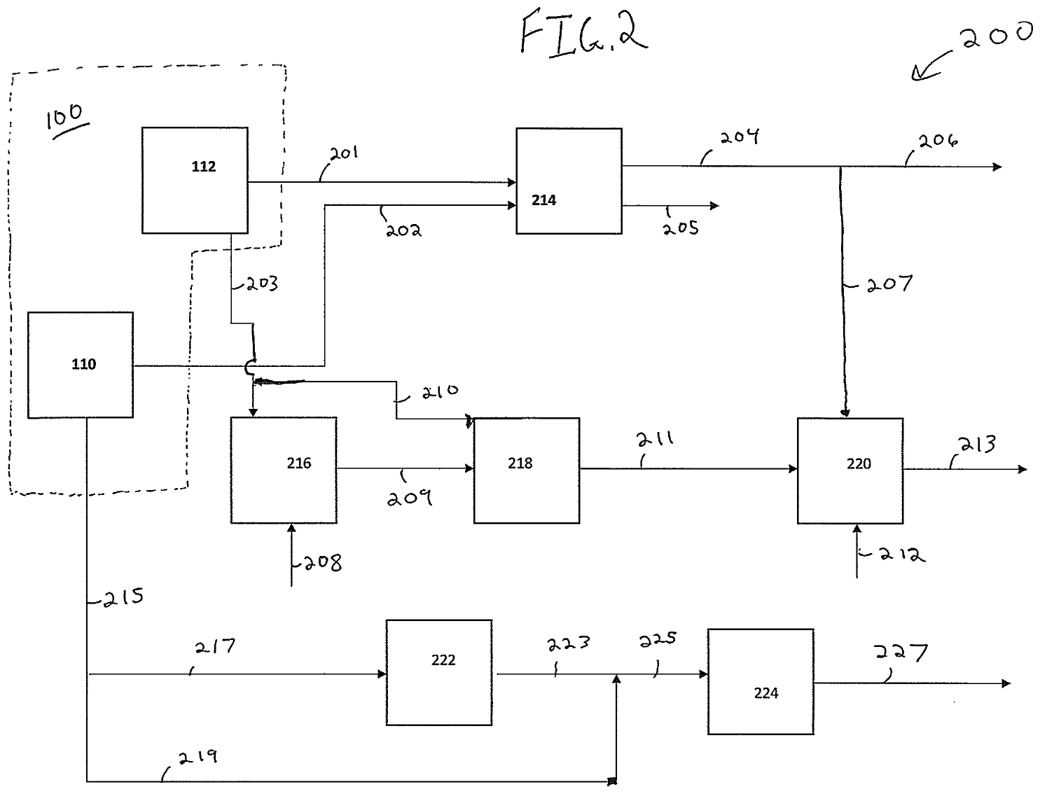

FIG. 2 depicts an illustrative integrated ammonia-urea-UAN system 200, according to one or more embodiments. The ammonia-urea-UAN system 200 can include the ammonia system 100 as described herein and in FIG. 1. The ammonia-urea-UAN system 200 can further include a urea plant 214 in fluid communication with the ammonia system 100, a nitric acid unit 216 in fluid communication with the ammonia system 100, an ammonium nitrate unit 218 in fluid communication with the nitric acid unit 216, a UAN unit 220 in fluid communication with a water source, the ammonium nitrate unit 218 and the urea plant 214, an amine unit 222 in fluid communication with the ammonia system 100, and a carbon dioxide compressor 224 in fluid communication with the amine unit 222.

In one or more embodiments, the urea plant 214 can be or include any suitable urea production unit, system, or facility for providing urea from ammonia and carbon dioxide. For example, the urea plant 214 can include one or more compressors and one or more heaters for compressing and heating a feed gas mixture of the ammonia and carbon dioxide. The urea plant 214 can also include a urea synthesis reactor. In one or more embodiments, the urea synthesis reactor can contain a catalyst bed. The catalyst bed suitable for urea synthesis can include metal oxides, mixed metal oxides, and supported metal catalysts. In one or more embodiments, the catalyst bed can be or include iron oxide supported on a high-surface-area material, such as alumina. In one or more embodiments, the catalyst bed can include a mixed metal oxide catalyst, such as a combination of iron, chromium, and copper, for example. Other suitable catalysts for use in urea synthesis include zeolite-based catalysts and nanostructured catalysts.

In operation of the urea plant 214, at least a portion of the ammonia product provided by the ammonia system 100, for example the ammonia product in line 131, can be introduced to the urea plant 214 via line 201. In addition, at least a portion of the carbon dioxide provided by the syngas generation system 110, for example the carbon dioxide stream in line 125, can be introduced to the urea plant 214 via line 202. The ammonia in line 201 and the carbon dioxide in line 202 can be mixed prior to or within the urea plant 214 to provide a urea feed gas mixture. The urea feed gas mixture can then be compressed and heated. For example, the urea feed gas mixture can be compressed to a pressure of between about 100 and 300 bar and preheated to a temperature of between about 150 and 250° C. to provide a preheated urea feed gas mixture. The preheated urea feed gas mixture can then be introduced to the urea synthesis reactor, which can contain one or more of the catalyst beds described herein.

The preheated urea feed gas mixture can then be subjected to the urea synthesis reaction in the presence of the catalyst bed. The reaction can occur at a temperature of between about 160 and 220° C. and a pressure of between about 150 and 250 bar. As the preheated urea feed gas mixture passes through the catalyst bed, urea is formed and is subsequently separated from the reaction mixture to provide liquid urea in line 204 and water or the remaining aqueous solution in line 205. The liquid urea in line 204 can be subjected to a pelletizing step, such as a prilling tower, to obtain prilled urea. In one or more embodiments, at least a portion of the liquid urea in line 204 can be introduced to UAN unit 220, as described herein, via line 207. The remaining liquid urea, for example in line 206, can be sent to a prilling tower or similar apparatus to provide prilled urea product.

In one or more embodiments, the nitric acid unit 216 can be or include any unit or system suitable for producing nitric acid from a reaction of air with ammonia, which can take place in a reactor. The reactor of the nitric acid unit 216 can include a catalytic bed containing a catalyst, such as a platinum or rhodium catalyst, supported on a porous carrier.

In operation of the nitric acid unit 216, at least a portion of the ammonia product provided by the ammonia system 100, for example, the ammonia product in line 131, can be introduced to the nitric acid unit 216 via line 203. In one or more embodiments, air via line 208 can also be introduced to the nitric acid unit 216. The ammonia and air can then be mixed in the presence of the catalyst in the reactor of the nitric acid unit 216. The mixture can then be heated, for example to a temperature between 800 and 1000° C., for a reaction between the ammonia and oxygen from the air to occur, resulting in the formation of nitrogen oxides, which can then be absorbed in water to produce nitric acid via line 209. The nitric acid in line 209 can then be introduced to the ammonium nitrate unit 218.

In one or more embodiments, the ammonium nitrate unit 218 can be or include any unit or system suitable for converting nitric acid and ammonia to ammonium nitrate. In operation, nitric acid via line 209 and ammonia via line 210 can be introduced to the ammonium nitrate unit 218. The ammonium nitrate unit 218 can be operated under conditions sufficient to form an aqueous ammonium nitrate solution by reacting the nitric acid with the ammonia. In one or more embodiments, the nitric acid in line 209 can be in a concentration of about 20 wt % to about 80 wt %. The ammonium nitrate unit 218 can include a neutralizer reactor. The neutralizer reactor can be or include a U-type combination of a circular tube and mixing tube with a separation vessel reactor. The neutralizer reactor can be operated at a pressure of about 0.05 MPa, about 0.1 MPa, or about 0.125 MPa to about 0.175 MPa, about 0.2 MPa, or about 0.5 MPa and a temperature of about 100° C., about 115° C., or about 135° C. to about 150°° C., about 165° C. or about 175° C. The produced ammonium nitrate can be withdrawn from the ammonium nitrate unit 218 via line 211. The ammonium nitrate in line 211 can be a solution having an ammonium nitrate concentration of about 70% to 95%, such as 83% to 88%, for example with pH of about 7. The ammonium nitrate solution in line 211 can then be introduced to the UAN unit 220.

In one or more embodiments, the UAN unit 220 can be or include a mixing unit, such as a static mixer or pipe mixer, for example. In operation, the ammonium nitrate solution in line 211, water in line 212, and the liquid urea in line 207 can be introduced to and mixed or otherwise combined in the UAN unit 220 to provide an aqueous solution of UAN in line 213. The aqueous solution of UAN can be withdrawn from the UAN unit 220 as a UAN product via line 213. The UAN product in line 213 can include about 20 wt %, about 25 wt %, or about 28 wt % to 32 wt %, about 35 wt %, or about 40 wt % of total nitrogen and from about 25 wt %, 27 wt %, or about 29 wt % to about 35 wt %, about 38 wt %, or about 40 wt % urea and of from about 32 wt %, about 34 wt %, or about 36 wt % to about 45 wt %, about 48 wt %, or about 50 wt % ammonium nitrate, with the remainder being water.

In one or more embodiments, the power plant carbon dioxide containing stream in line 113 can be mixed or otherwise combined with carbon dioxide-containing stream in line 125 to provide a combined carbon dioxide-containing stream via line 215. In one or more embodiments, the combined carbon dioxide-containing stream in line 215 can be or include all the carbon dioxide provided by the syngas generation system 110 that is not otherwise introduced to the urea plant 214 (e.g., all remaining carbon dioxide). In one or more embodiments, at least a portion of the combined carbon dioxide-containing stream in line 215 can be introduced one or more amine absorption unit(s) 222 via line 217 to provide a pure carbon dioxide stream in line 223.

The one or more amine absorption unit(s) 222 can include any unit(s) suitable for removing carbon dioxide from gases. Suitable amine absorption units include but are not limited to units suitable for contacting a carbon dioxide containing gaseous stream with a liquid absorbing fluid to provide a pure carbon dioxide stream having a carbon dioxide concentration of about at least about 90 wt %, at least about 95 wt %, at least about 98 wt %, at least about 99 wt %, at least about 99.5 wt %, or at least about 99.9 wt %. In one or more embodiments, the liquid absorbing fluid can be or include one or more of-methyldiethanolamine (MDEA), an aqueous solution of triethanolamine, an aqueous solution of potassium carbonate, or an aqueous solution of tertiary alkylamines selected from diamines, triamines and the like.

In one or more embodiments, at least a portion of the combined carbon dioxide-containing stream in line 215 can bypass the amine absorption unit 222. For example, if the combined carbon dioxide-containing stream in line 215 is pure or has a carbon dioxide concentration of at least about 90 wt %, at least about 95 wt %, at least about 98 wt %, at least about 99 wt %, at least about 99.5 wt %, or at least about 99.9 wt % then the combined carbon dioxide-containing stream in line 215 can bypass the amine absorption unit 222 via line 219. The pure carbon dioxide stream in line 223 can optionally be combined with the carbon dioxide in line 219 to provide a pure combined carbon dioxide stream in line 225. The pure combined carbon dioxide stream in line 225 can then be sent to a compression unit 224 to provide a compressed carbon dioxide stream via line 227. In one or more embodiments (not shown), the carbon dioxide in line 219 and the pure carbon dioxide stream in line 223 can be independently sent to the compression unit 224 to provide the compressed carbon dioxide. In one or more embodiments, the compressed carbon dioxide via line 227 can be introduced to one or more wells or other subterranean storage means suitable for sequestration of the carbon dioxide. In one or more embodiments, the carbon dioxide via line 227 can be sequestered by reacting the carbon dioxide with metal oxides to provide carbonates.

In one or more embodiments, the carbon dioxide via line 227 can be monitored to correlate hydrogen byproduct produced with the carbon dioxide that is ultimately stored or sequestered to ensure such hydrogen byproduct is blue hydrogen or otherwise appropriately designated as blue hydrogen. The designation of the hydrogen in line 133 and/or the ammonia in line 201 as grey, blue, or green, could be used to adjust the amount of green hydrogen supplied to the ammonia synthesis unit 112. For example, the amount of produced carbon dioxide released to the atmosphere in the production of the hydrogen in line 129 could determine the amount of green hydrogen in line 127 is blended therewith to provide the blended hydrogen in line 133. In one or more embodiments, the overall carbon dioxide emissions footprint (e.g,, the carbon intensity) used to provide the hydrogen in line 133 and/or the ammonia in line 201 can be analyzed and used to determine the volumetric ratio of one or more of the blue-green hydrogen blend and/or the grey-green hydrogen blend in line 133, the blue-green ammonia blend and/or grey-green ammonia blend in line 201. The carbon intensity can be calculated by any number of methods, including but not limited to the GREET Model to calculate carbon intensities of fuels produced by various processes developed by Argonne National Labs.

The blending ratio(s) may be selected based on one or more carbon credits programs and/or green hydrogen production credit programs (together referred to herein as “green credits”). Such green credits can be or include any rights, credits, revenues, offsets, greenhouse gas rights, or similar rights related to carbon credits, rights to any greenhouse gas emission reductions, carbon-related credits or equivalent arising from emission reduction trading or any quantifiable benefits, whether created from or through a governmental authority, a private contract, or otherwise. In one or more embodiments, the green credits can be or include the clean hydrogen production tax credit in the Inflation Reduction Act of 2022 (“IRA”). For example, under that credit system when a ratio of kilogram of carbon dioxide emitted per kilogram of hydrogen produced is between 2.5 to 4.0, then the credit value is $0.60 per kilogram of hydrogen produced, if the ratio is 1.5 to 2.4, the credit is $0.75 per kilogram of hydrogen produced, if the ratio is 0.5 to 1.4, the credit is $1.00 per kilogram of hydrogen produced, and if the ratio is less than 0.5, the credit is $3.00 per kilogram of hydrogen produced. In practice, the cost of generating grey, blue, and green hydrogen may fluctuate, while at the same time, the clean hydrogen production tax credits or other similar credit system may be change over time or depend on the territory receiving the final product, resulting in the need for a flexible blending system that can efficiently and swiftly balance costs of generating a blended hydrogen stream and/or blended ammonia stream based on any applicable tax credit-like scheme.

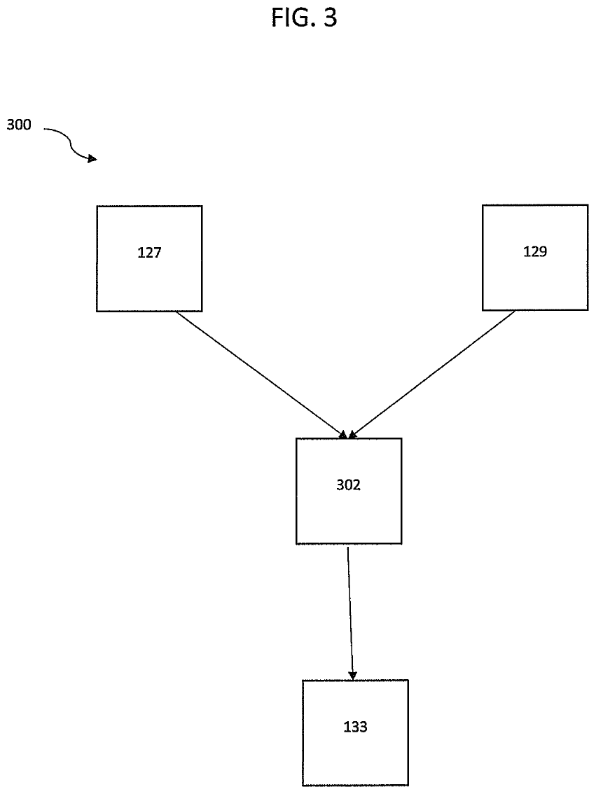

FIG. 3 depicts an illustrative flow chart 300 of at least one embodiment of a method for blending grey, blue, and/or green hydrogen, which may be performed by one or more components of the system of FIGS. 1 and 2, as described herein. In one or more embodiments, the blended hydrogen in line 133 can be provided by blending the green hydrogen stream in line 127 with the grey or blue hydrogen stream in line 129 via a blending unit 302. The blending unit 302 can be or include any apparatus suitable for blending the green hydrogen stream in line 127 with the grey or blue hydrogen stream in line 129—at a desired blend ratio to yield the hydrogen blend in line 133.

The blending unit 302 can include a first hydrogen flow sensor in sensory communication with the green hydrogen stream in line 127, a second hydrogen flow sensor in sensory communication with the grey or blue hydrogen stream in line 129, and a remote information processing unit in informational communication with the first and second hydrogen flow sensors, for remotely monitoring and displaying hydrogen flow rates detected by the first and second hydrogen flow sensors.

In one or more embodiments, the blending unit 302 can include at least two valves (not shown). One valve can be an on/off valve located between the green hydrogen stream in line 127 and the grey and/or blue hydrogen stream in line 129. This valve can prevent grey and/or blue hydrogen from entering the blending unit 302. The second valve can be a modulating valve that controls the flow of green hydrogen towards the first valve. The second valve can control the rate of flow of green hydrogen by modulating both the pressure of the green hydrogen stream passing through the valve as well as the size of the orifice through which the green hydrogen stream flows. The modulating valve and/or the on/off valve can be under the control of a process control unit, which can vary or adjust the blend ratio to attain a desired concentration of green hydrogen in line 133, based on the flow rate of the green hydrogen entering the blending unit, the flow rate of grey and/or blue hydrogen entering the blending unit 302, and the desired flow rate of the blended hydrogen in line 133. A green hydrogen addition rate may then be calculated based upon the blend ratio and the rate of flow in the grey and/pr blue hydrogen stream, and the modulating valve may be opened or closed to allow green hydrogen addition at the rate thus calculated. The valves may also be under the control of one or more remote information processing units (e.g., via the controller as described herein) contained in the process control unit.

In one or more embodiments, the process control unit can include a controller, one or more informational databases, and an information processing unit (IPU). The one or more informational databases and the IPU can be configured to store green credit data (e.g., the knowledge of all green credits, including their respective value, applicable to the ammonia, urea, and/or UAN produced according to the methods described herein) and production data, including carbon intensity data, corresponding to the production of the grey and/or blue hydrogen, and then calculating and sending blending instructions based upon such data. The production data can also include one or more of natural gas market price information, grey, green, and blue hydrogen market price information, urea market price information, grey, green, and blue ammonia market price information, and UAN market price information. In such embodiments, the informational database can store the green credit data and the production data for the grey and/or blue hydrogen stream in line 129. The production data for the grey and/or blue hydrogen stream in line 129 can include the amount of carbon dioxide resulting from the production of the hydrogen byproduct in line 129 that is ultimately captured as described herein. The IPU can then retrieve the green credit data and the production data for the grey and/or blue hydrogen stream in line 129 from the informational database. The IPU can then calculate the blend ratio and/or blend rate based upon one or more of the costs to produce the green hydrogen in line 127, the value of the available green credits, and the amount of carbon dioxide captured in the production of the grey and/or blue hydrogen in line 129. As described herein, the term “retrieve” includes both retrieving data and receiving data from another source.

In one or more embodiments, the green credit data can be a computed estimate of credits that can be earned using the systems and methods described herein. For example, the green credits can be calculated by estimating the amount of carbon dioxide captured for the grey and/or blue hydrogen byproduct in line 129 and/or the amount of green hydrogen in line 127 introduced to the blending unit 302 and applying one or more applicable green credits, which can fluctuate depending on applicable laws, to the estimated blended hydrogen in line 133 to provide a green credit estimate. Once the green credit estimate is obtained, the green credit estimate can be inputted to the informational database for retrieval by the IPU. The IPU can then calculate an optimized blend ratio and/or blend rate based upon the cost to produce the green hydrogen in line 127, the green credit estimate, and the amount of carbon dioxide captured in the production of the grey and/or blue hydrogen in line 129. Any suitable controller or control unit can then adjust the blending ratio settings in the blending unit 302 based on the calculated optimized blend ratio and/or blend rate determined by the IPU. In one or more embodiments, the blending ratio based on the calculated optimized blend ratio and/or blend rate determined by the IPU can be adjusted manually.

In one or more embodiments, the IPU can include an information platform for integrating additional green credit information into analysis for operating the systems and methods described herein. For example, the IPU can include one or more machine learning tools for curating data sources relating to green credit data, production data, including carbon intensity data, corresponding to the production of the grey and/or blue hydrogen, and then calculating and sending blending instructions based upon such data, natural gas market price information, grey, green, and blue hydrogen market price information, urea market price information, grey, green, and blue ammonia market price information, and UAN market price information, and incorporating the data sources into the informational database for retrieval by the IPU to provide an optimized blend ratio determined by the IPU. Example methods and systems for incorporating AI moderated information sources are described in U.S. Pre-Grant Publication No. 2021/0256084 A1, which is incorporated herein by reference.

In one or more alternative embodiments, the flow chart 300 for blending grey, blue, and/or green hydrogen as described herein, can be adapted for blending green, blue, and/or grey ammonia to provide the ammonia in line 201.

As used herein, the terms “inner” and “outer”; “up” and “down”; “upper” and “lower”; “upward” and “downward”; “above” and “below”; “inward” and “outward”; “uphole” and “downhole”; and other like terms as used herein refer to relative positions to one another and are not intended to denote a particular direction or spatial orientation. The terms “couple,” “coupled,” “connect,” “connection,” “connected,” “in connection with,” and “connecting” refer to “in direct connection with” or “in connection with via one or more intermediate elements or members.”

The foregoing has outlined features of several embodiments so that those skilled in the art may better understand the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method for producing ammonia, comprising:
providing a hydrocarbon feed stock and an oxygen-containing stream;
reacting the hydrocarbon feed stock with the oxygen-containing stream to provide a syngas containing a carbon dioxide stream and a first hydrogen stream;
electrolyzing water to provide a green hydrogen stream;
blending the first hydrogen stream and the green hydrogen stream in a blending unit to provide a blended hydrogen stream;
introducing the blended hydrogen stream to an ammonia synthesis system to provide an ammonia product.

2. The method of claim 1, wherein the first hydrogen stream is grey hydrogen and the ammonia product is grey-green ammonia.

3. The method of claim 1, wherein the first hydrogen stream is blue hydrogen and the ammonia product is blue-green ammonia.

4. The method of claim 1, wherein the blending unit comprises a first valve and a second valve, wherein the first valve is an on/off valve located between the green hydrogen and the first hydrogen stream, and wherein the second valve is a modulating valve configured to control the flow of green hydrogen towards the first valve.

5. The method of claim 4, wherein the modulating valve and/or the on/off valve are controlled by a process control unit that is configured to adjust a blend ratio of green hydrogen to the first hydrogen to attain a desired concentration of green hydrogen in the blended hydrogen stream.

6. The method of claim 5, wherein the process control unit comprises a database containing green credit data and production data relating to the production of the first hydrogen stream.

7. The method of claim 1, wherein the blended ammonia product is reacted with at least a portion the carbon dioxide stream to provide a urea product.

8. The method of claim 7, wherein another portion of the carbon dioxide stream is sequestered, and the amount of carbon dioxide to be sequestered is determined by the green credit data and/or production data via the process control unit.

9. A method for producing urea, comprising:
providing a hydrocarbon feed stock and an oxygen-containing stream;
reacting the hydrocarbon feed stock with the oxygen-containing stream to provide a syngas containing a carbon dioxide stream and a first hydrogen stream;
electrolyzing water to provide a green hydrogen stream;
blending the first hydrogen stream and the green hydrogen stream in a blending unit to provide a blended hydrogen stream;
introducing the blended hydrogen stream to an ammonia synthesis system to provide an ammonia product; and
introducing at least a portion of the carbon dioxide stream and the ammonia product to a urea plant to provide liquid urea.

10. The method of claim 9, wherein the liquid urea is pelletized to provide prilled urea.

11. The method of claims 9, wherein the blending unit comprises:
a first hydrogen source for generating the first hydrogen;
a green hydrogen source for generating green hydrogen;
a blending unit for blending the blue hydrogen and the green hydrogen to produce blended hydrogen;
a processing control unit comprising:
an information processing unit for determining a desired blending ratio of the blue hydrogen and the green hydrogen based on hydrogen credit availability; and
a controller for adjusting the blend ratio of the blue hydrogen and the green hydrogen produced by the blending unit based on the desired blend ratio determined by the information processing unit to optimize the use of hydrogen credits.

12. The method of claim 11, wherein a modulating valve and/or an on/off valve are controlled by the process control unit that is configured to adjust the blend ratio to attain a desired concentration of green hydrogen in the blended hydrogen stream.

13. The method of claim 12, wherein the process control unit comprises a database containing green credit data and production data relating to the production of the first hydrogen stream.

14. The method of claim 13, wherein another portion of the carbon dioxide stream is sequestered, and the amount of carbon dioxide to be sequestered is determined by the green credit data and/or production data via the process control unit.

15. The method of claim 14, wherein the portion of the carbon dioxide stream that is sequestered is all of the carbon dioxide from the carbon dioxide stream that is not introduced to the urea plant.

16. A method for producing UAN, comprising:
providing a hydrocarbon feed stock and an oxygen-containing stream;
reacting the hydrocarbon feed stock with the oxygen-containing stream to provide a syngas containing a carbon dioxide stream and a first hydrogen stream;
electrolyzing water to provide a green hydrogen stream;
blending the first hydrogen stream and the green hydrogen stream in a blending unit to provide a blended hydrogen stream;
introducing the blended hydrogen stream to an ammonia synthesis system to provide an ammonia product;
introducing a first portion of the carbon dioxide stream and a first portion of the ammonia product to a urea plant to provide liquid urea;
introducing a second portion of the ammonia product and air to a nitric acid unit to provide a nitric acid stream;
introducing a third portion of the ammonia product and the nitric acid stream to an ammonium nitrate unit to provide an ammonium nitrate solution; and
introducing the ammonium nitrate solution and at least a portion of the liquid urea to provide a mixer to provide a UAN product.

17. The method of claim 16, wherein the blending unit comprises:
a first hydrogen source for generating the first hydrogen;
a green hydrogen source for generating green hydrogen;
a blending unit for blending the blue hydrogen and the green hydrogen to produce blended hydrogen;
a processing control unit comprising:
an information processing unit for determining a desired blending ratio of the blue hydrogen and the green hydrogen based on hydrogen credit availability; and
a controller for adjusting the blend ratio of the blue hydrogen and the green hydrogen produced by the blending unit based on the desired blend ratio determined by the information processing unit to optimize the use of hydrogen credits.

18. The method of claim 17, wherein a modulating valve and/or an on/off valve are controlled by the process control unit that is configured to adjust the blend ratio to attain a desired concentration of green hydrogen in the blended hydrogen stream.

19. The method of claim 18, wherein the process control unit comprises a database containing green credit data and production data relating to the production of the first hydrogen stream.

20. The method of claim 19, wherein another portion of the carbon dioxide stream is sequestered, and the amount of carbon dioxide to be sequestered is determined by the green credit data and/or production data via the process control unit.
